(19) United States
(12) Patent Application Publication
(10) Pub. No.: US 2025/0349047 A1
(43) Pub. Date: Nov. 13, 2025

(54) SYSTEM AND METHOD FOR AUTOMATICALLY DETERMINING IMAGE SIZE

(71) Applicant: GE Precision Healthcare LLC, Waukesha, WI (US)

(72) Inventors: Brian Edward Nett, Wauwatosa, WI (US); Arka Datta, Stockholm (SE); John Edward Londt, Oconomowoc, WI (US); Jasper Kim Ocampo, Sussex, WI (US); Jiahua Fan, New Berlin, WI (US)

(21) Appl. No.: 18/660,361

(22) Filed: May 10, 2024

(51) Int. Cl. G06T 11/00

(52) U.S. Cl. CPC G06T 11/005; G06T 2210/41

(57) ABSTRACT

A system and a method include obtaining, at a processor, a clinical task for a scan of a subject with a computed tomography imaging system. The systems and method also include obtaining, at the processor, scanning parameters for the scan. The system and method further include automatically determining, via the processor, reconstruction matrix parameters for generating a reconstructed image from tomographic data obtained of the subject with the scan based at least on the clinical task and the scanning parameters.

### BACKGROUND

The subject matter disclosed herein relates to medical imaging systems and, more particularly, to automatically determining an image size for tomographic data acquired with a computed tomography (CT) imaging system.

In CT, X-ray radiation spans a subject of interest, such as a human patient, and a portion of the radiation impacts a detector where the image data is collected. In digital X-ray systems a photodetector produces signals representative of the amount or intensity of radiation impacting discrete pixel regions of a detector surface. The signals may then be processed to generate an image that may be displayed for review. In the images produced by such systems, it may be possible to identify and examine the internal structures and organs within a patient's body. In CT imaging systems a detector array, including a series of detector elements or sensors, produces similar signals through various positions as a gantry is displaced around a patient, allowing volumetric reconstructions to be obtained.

A high resolution CT scanner utilizes a large image matrix size to provide an improved spatial resolution experience. These large image matrix sizes offer improved image quality in some situations via increased resolution at the expense of increased disk space utilization.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a computer-implemented method is provided. The computer-implemented method includes obtaining, at a processor, a clinical task for a scan of a subject with a computed tomography imaging system. The computer-implemented method also includes obtaining, at the processor, scanning parameters for the scan. The computer-implemented method further includes automatically determining, via the processor, reconstruction matrix parameters for generating a reconstructed image from tomographic data obtained of the subject with the scan based at least on the clinical task and the scanning parameters.

In another embodiment, a system is provided. The system includes a memory encoding processor-executable routines. The system also includes a processor configured to access the memory and to execute the processor-executable routines, wherein the processor-executable routines, when executed by the processor, cause the processor to perform actions. The actions include obtaining a clinical task for a scan of a subject with a computed tomography imaging system. The actions also include obtaining scanning parameters for the scan. The actions further include automatically determining reconstruction matrix parameters for generating a reconstructed image from tomographic data obtained of the subject with the scan based at least on the clinical task and the scanning parameters.

In a further embodiment, a non-transitory computer-readable medium, the computer-readable medium including processor-executable code that when executed by a processor, causes the processor to perform actions. The actions include obtaining a clinical task for a scan of a subject with a computed tomography imaging system. The actions also include obtaining scanning parameters for the scan. The actions further include automatically determining reconstruction matrix parameters for generating a reconstructed image from tomographic data obtained of the subject with the scan based at least on the clinical task and the scanning parameters.

### BRIEF DESCRIPTION OF THE DRAWINGS

These and other features, aspects, and advantages of the present disclosed subject matter will become better understood when the following detailed description is read with reference to the accompanying drawings in which like characters represent like parts throughout the drawings, wherein:

FIG. 1 is a pictorial representation of a CT imaging system (e.g., having a light detection and ranging (LiDAR) scanning system), in accordance with aspects of the present disclosure;

FIG. 2 is a block diagram of the CT imaging system in FIG. 1, in accordance with aspects of the present disclosure;

FIG. 3 is a pictorial representation of a CT imaging system and an optical imaging system, in accordance with aspects of the present disclosure;

FIG. 4 is a schematic diagram of a scan room having a CT imaging system and optical imaging system (e.g., camera on scanner), in accordance with aspects of the present disclosure;

FIG. 5 is a flowchart of a method for reconstructing CT imaging data, in accordance with aspects of the present disclosure;

FIG. 6 is a flowchart of another method for reconstructing CT imaging data, in accordance with aspects of the present disclosure;

FIG. 7 is a flowchart of a further method for reconstructing CT imaging data, in accordance with aspects of the present disclosure;

FIG. 8 is a flowchart of a method for determining a reconstruction field of view (e.g., utilizing initial reconstructed image), in accordance with aspects of the present disclosure;

FIG. 9 depicts an example of an initial reconstructed CT image, in accordance with aspects of the present disclosure;

FIG. 10 depicts an example of a subsequent reconstructed CT image, in accordance with aspects of the present disclosure;

FIG. 11 is a flowchart of a method for determining a reconstruction field of view (e.g., utilizing surface map from LiDAR data), in accordance with aspects of the present disclosure;

FIG. 12 depicts an example of a 2D contour representation for a surface of a region of interest of a subject generated from LiDAR data, in accordance with aspects of the present disclosure;

FIG. 13 is a flowchart of a method for determining a reconstruction field of view (e.g., utilizing a body contour), in accordance with aspects of the present disclosure;

FIG. 14 depicts an example of a lookup table for determining matrix size, in accordance with aspects of the present disclosure;

FIG. 15 depicts an example of a user interface or conducting primary reconstruction utilizing the automated image size feature, in accordance with aspects of the present disclosure; and

FIG. 16 depicts an example of a user interface for conducting secondary reconstruction utilizing the automated image size feature, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion may be provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as tomographic image reconstruction for industrial Computed Tomography (CT) used in non-destructive inspection of manufactured parts or goods (i.e., quality control or quality review applications), and/or the non-invasive inspection of packages, boxes, luggage, and so forth (i.e., security or screening applications). In general, the present approaches may be useful in any imaging or screening context to provide automatically an optimal matrix or image size.

The present disclosure provides systems and methods for automatically determining reconstruction matrix parameters for generating a reconstructed image of optimal size. Automatically determining the optimal image matrix size enables a user to benefit from using larger matrix sizes while minimizing their disk space utilization, thus, optimizing resource utilization. In particular, the disclosed systems and methods enable the utilization of a higher resolution CT scanner while not always generating large images. In addition, the disclosed systems and methods enable faster reconstruction. Further, the disclosed systems and methods can improve both the workflow and resource management without impacting image quality.

The systems and methods include obtaining a clinical task for a scan of a subject with a computed tomography imaging system. In certain embodiments, the clinical task is obtained via user input. In certain embodiments, obtaining from a hospital information system or radiology information system. The systems and methods also include obtaining scanning parameters for the scan. The disclosed systems and methods further include automatically determining reconstruction matrix parameters for generating a reconstructed image from tomographic data obtained of the subject with the scan based at least on the clinical task and the scanning parameters.

In certain embodiments, automatically determining the reconstruction matrix parameters includes automatically determining, via the processor, a reconstruction field of view. In certain embodiments, automatically determining the reconstruction field of view includes obtaining initial tomographic data of the subject with the computed tomography imaging system; performing full field of view reconstruction on the initial tomographic data to generate an initial reconstructed image, wherein the initial reconstructed image has a lower resolution (e.g., lower image quality) than a reconstructed image of the tomographic data generated utilizing the reconstruction matrix parameters (as often the first reconstruction maybe very fast to save time and use a lower matrix size); and automatically determining the reconstruction field of view based on the initial reconstructed image.

In certain embodiments, automatically determining the reconstruction field of view includes obtaining, at the processor, light detection and ranging (LiDAR) data of the subject acquired with a LiDAR scanning system coupled to a gantry of the computed tomography imaging system; generating, via the processor, a surface map of the subject based on the LiDAR data; and automatically determining, via the processor, the reconstruction field of view based on the surface map.

In certain embodiments, automatically determining the reconstruction field of view includes obtaining, at the processor, imaging data of the subject acquired with a three-dimensional camera coupled to a gantry of the computed tomography imaging system; generating, via the processor, a body contour of the subject based on the imaging data; and automatically determining, via the processor, the reconstruction field of view based on the body contour.

In certain embodiments, automatically determining the reconstruction matrix parameters includes automatically determining, via the processor, a matrix size based at least on the clinical task, the scanning parameters, and the reconstruction field of view. In certain embodiments, automatically determining the matrix size includes utilizing, via the processor, a lookup table to determine the matrix size.

In certain embodiments, the disclosed systems and methods include obtaining, at the processor, additional selected parameters that influence the matrix size, wherein the matrix size is automatically determined based on the clinical task, the scanning parameters, the reconstruction field of view, and the additional selected parameters. In certain embodiments, automatically determining the matrix size includes calculating, via the processor, the matrix size based on one or more of the scanning parameters and the additional selected parameters. In certain embodiments, the additional selected parameters are obtained via user input. In certain embodiments, the additional selected parameters are automatically determined, via the processor, based on the obtained clinical task. In certain embodiments, the additional selected parameters comprise reconstruction kernel, iterative reconstruction, and post processing filters.

In certain embodiments, the disclosed systems and methods include automatically updating a reconstruction prescription to include the reconstruction field of view and the matrix size. In certain embodiments, the disclosed systems and methods include generating a reconstructed image utilizing the updated reconstruction prescription.

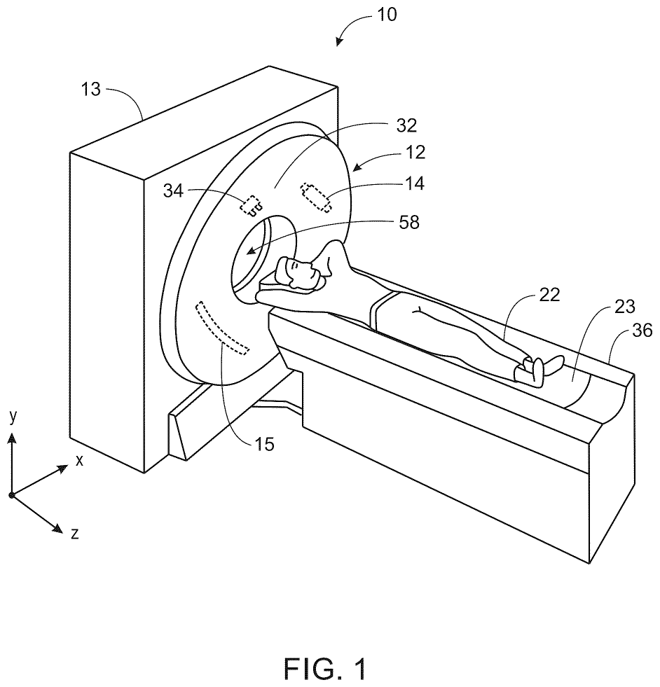

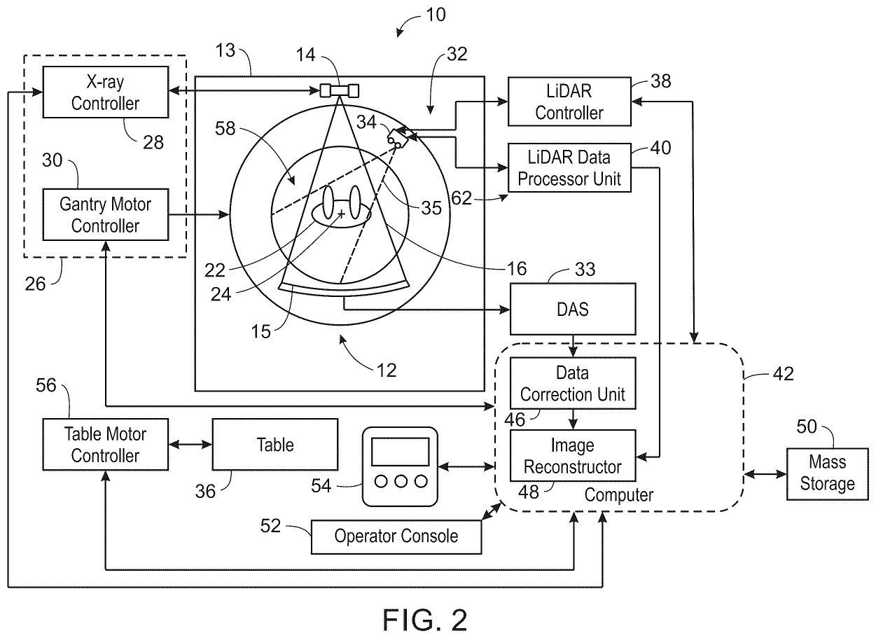

With the preceding in mind and referring to FIGS. 1 and 2, a CT imaging system 10 is shown, by way of example. The CT imaging system 10 includes a gantry 12 coupled to a housing 13 (e.g., gantry housing). The gantry 12 has a rotating component and a stationary component. The gantry 12 has an X-ray source 14 that projects a beam of X-rays 16 toward an X-ray detector assembly or X-ray detector array 15 (e.g., having a plurality of detector modules) on the opposite side of the gantry 12. The X-ray source 14 and the X-ray detector assembly 15 are disposed on the rotating portion of the gantry 12. The X-ray detector assembly 15 is coupled to data acquisition systems (DAS) 33. The plurality of detector modules of the X-ray detector assembly 15 detect the projected X-rays that pass through a patient or subject 22 (disposed on a cradle 23 of a table 36), and DAS 33 converts the data to digital signals for subsequent processing. Each detector module of the X-ray detector assembly 15 in a conventional system produces an analog electrical signal that represents the intensity of an incident X-ray beam and hence the attenuated beam as it passes through the patient 22. During a scan to acquire X-ray projection data, gantry 12 and the components mounted thereon rotate about a center of rotation 24 (e.g., isocenter) so as to collect attenuation data from a multitude of view angles relative to the imaged volume.

Rotation of gantry 12 and the operation of X-ray source 14 are governed by a control mechanism 26 of CT imaging system 10. Control mechanism 26 includes an X-ray controller 28 that provides power and timing signals to the X-ray source 14 and a gantry motor controller 30 that controls the rotational speed and position of gantry 12.

In certain embodiments, the imaging system 10 also includes a light detection and ranging (LiDAR) scanning system 32 physically coupled to the imaging system 10. The LiDAR scanning system 32 includes one or more LiDAR scanners or instruments 34. As depicted, the LiDAR scanning system 32 has one LiDAR scanner 34. The one or more LiDAR scanners 34 are utilized to acquire depth dependent information (LiDAR data or light images) of the patient 22 with high spatial fidelity. The depth dependent information is utilized in subsequent workflow processes for a CT scan. The one or more LiDAR scanners 34 emit pulsed light 35 (e.g., laser) at the patient 22 and detect the reflected pulsed light from the patient 22. The LiDAR scanning system 32 is configured to acquire the LiDAR data from multiple different views (e.g., at different angular positions relative to the axis of rotation 24).

In certain embodiments, as depicted in FIGS. 1 and 2, the LiDAR scanner 34 is coupled to the gantry 12. In particular, the LiDAR scanner 34 is disposed within the gantry housing 13 outside a scan window. The LiDAR scanner 34 is rotated across the patient 22 to acquire the LiDAR data at the different angular positions. In certain embodiments, multiple LiDAR scanners 34 may be coupled to the gantry 12 and rotated to acquire the LiDAR data at the different angular positions.

In certain embodiments, multiple LiDAR scanners 34 may be coupled to the gantry 12 in fixed positions but disposed at different angular positions (e.g., relative to axis of rotation 24). The LiDAR scanners 34 in fixed positions may acquire the LiDAR data at the same time while remaining stationary.

In certain embodiments, the LiDAR scanning system 32 may be external to the gantry 12 but still physically coupled to the imaging system 10. For example, multiple LiDAR scanners 34 may be coupled to a LiDAR panel (e.g., at different angular positions relative to the axis of rotation 24) that is coupled to a guide rail system. The guide rail system may be coupled to the gantry housing 13 or a table 36 of the system 10. The guide rail system may be configured to move the LiDAR panel toward and away from the gantry 12. In certain embodiments, the guide rail system may also be configured to rotate the LiDAR panel about the axis of rotation 24.

The LiDAR scanning system 32 includes a LiDAR controller 38 configured to provide timing and control signals to the one or more LiDAR scanners 34 for acquiring the LiDAR data at the different angular positions. The LiDAR data may be acquired prior to, during, and/or subsequent to a CT scan of the patient 22. The LiDAR scanning system 32 also includes a LiDAR data processing unit 40 that receives or obtains the LiDAR data from the one or more LiDAR scanners 34. The LiDAR data processing unit 40 utilizes time of flight information of the reflected pulsed light and processes the LiDAR data (e.g., acquired at the different views) to generate an accurate 3D measurement of the patient 22. The 3D measurement of the patient 22 has a high spatial resolution (e.g., sub mm accuracy). As noted above, the 3D measurement may be utilized in subsequent workflow processes of a CT scan as described in greater detail below.

The 3D measurement information from the LiDAR scanning system 32 (e.g., from the LiDAR data processing unit 40) and the scan data from the DAS 33 is input to a computer 42. The computer 42 also includes a data correction unit 46 for processing or correcting the CT scan data from the DAS 33. The computer 42 further includes an image reconstructor 48. The image reconstructor 48 receives sampled and digitized X-ray data from DAS 33 and performs high-speed reconstruction. The reconstructed image is applied as an input to the computer 42, which stores the image in a mass storage device 50. Computer 42 also receives commands and scanning parameters from an operator via console 52. An associated display 54 allows the operator to observe the reconstructed image as well as the 3D measurement data and other data from the computer 42. The operator supplied commands and parameters are used by computer 42 to provide control signals and information to the DAS 33, X-ray controller 28, gantry motor controller 30, and the LiDAR controller 38. In addition, computer 42 operates a table motor controller 56, which controls a motorized table 36 to position the patient 22 relative to the gantry 12. Particularly, table 36 moves portions of the patient 22 (via the cradle 23 that supports the patient 22) through a gantry opening or bore 58.

The computer 42 and the LiDAR processing unit 40 include may each include processing circuitry. The processing circuitry may be one or more general or application-specific microprocessors. The processing circuitry may be configured to execute instructions stored in a memory to perform various actions. For example, the processing circuitry may be utilized for receiving or obtaining LiDAR data acquired with the LiDAR scanning system 32. In addition, the processing circuitry may also generate a 3D measurement of the patient 22. Further, the processing circuitry may utilize the 3D measurement in a subsequent workflow process for a CT scan of the patient with the CT imaging system 32.

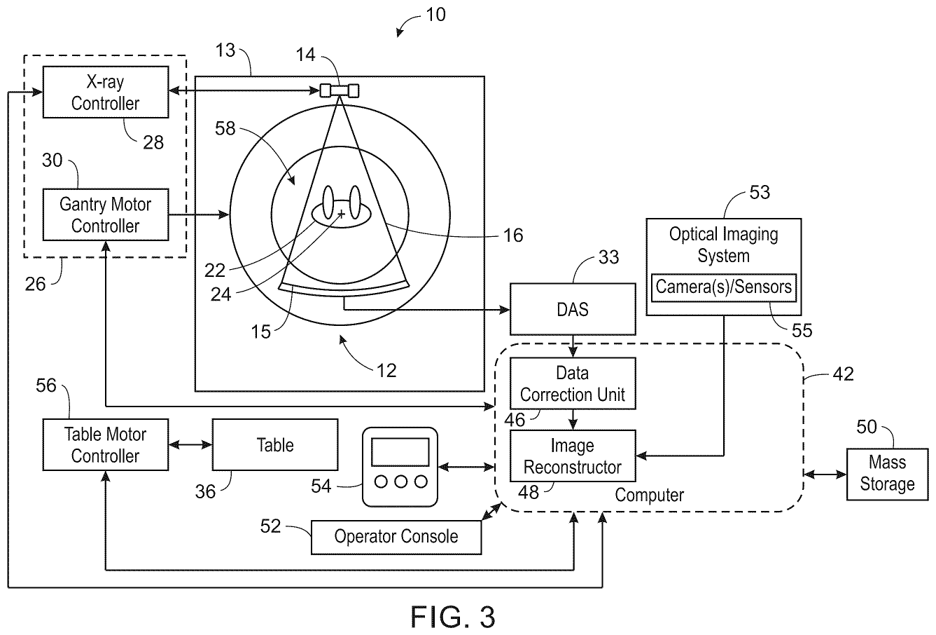

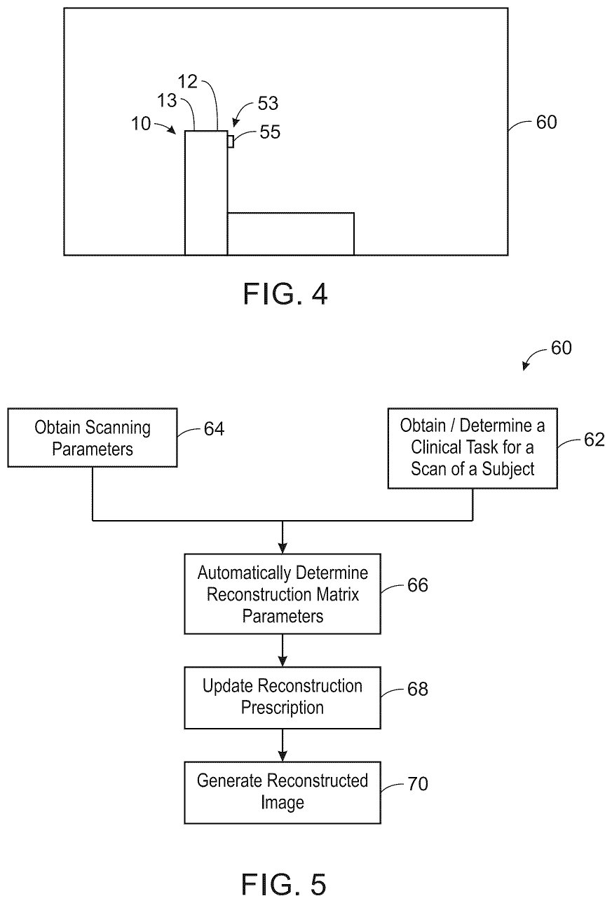

In certain embodiments, instead of a LiDAR scanning system, the CT imaging system 10 includes an optical imaging system 53 as depicted in FIG. 3. The optical imaging system 53 may include one or more cameras or sensors 55. In certain embodiments, the cameras or sensors 55 include a three-dimensional (3D) camera configured to acquired imaging data of the patient 22 that is utilized to generate or to determine a body contour of the patient. In certain embodiments, the one or more cameras 55 may be disposed at a top of housing of the gantry 12 of the CT imaging system 10 as depicted in FIG. 4. In certain embodiments, the camera 55 may be directly coupled to the gantry 12.

The processing circuitry of the CT imaging system 10 is configured to obtain a clinical task for a scan of a subject with CT imaging system 10. In certain embodiments, the clinical task is obtained via user input. In certain embodiments, obtaining from a hospital information system or radiology information system. The processing circuitry of the CT imaging system 10 is also configured to obtain scanning parameters for the scan. The processing circuitry of the CT imaging system 10 is further configured to automatically determine reconstruction matrix parameters for generating a reconstructed image from tomographic data obtained of the subject with the scan based at least on the clinical task and the scanning parameters.

In certain embodiments, the processing circuitry of the CT imaging system 10 is configured to automatically determine the reconstruction matrix parameters by automatically determining a reconstruction field of view. In certain embodiments, the processing circuitry of the CT imaging system 10 is configured to automatically determine the reconstruction field of view by obtaining initial tomographic data of the subject with the computed tomography imaging system. In certain embodiments, the processing circuitry of the CT imaging system 10 is configured to perform full field of view reconstruction on the initial tomographic data to generate an initial reconstructed image, wherein the initial reconstructed image has a lower resolution (e.g., lower image quality) than a reconstructed image of the tomographic data generated utilizing the reconstruction matrix parameters. In certain embodiments, the processing circuitry of the CT imaging system 10 is configured to automatically determine the reconstruction field of view based on the initial reconstructed image.

In certain embodiments, the processing circuitry of the CT imaging system 10 is configured to automatically determine the reconstruction field of view by obtaining light detection and ranging (LiDAR) data of the subject acquired with a LiDAR scanning system coupled to a gantry of the computed tomography imaging system. In certain embodiments, the processing circuitry of the CT imaging system 10 is configured to generate a surface map of the subject based on the LiDAR data. In certain embodiments, the processing circuitry of the CT imaging system 10 is configured to automatically determine the reconstruction field of view based on the surface map.

In certain embodiments, the processing circuitry of the CT imaging system 10 is configured to automatically determine the reconstruction field of view by obtaining, at the processor, imaging data of the subject acquired with a three-dimensional camera coupled to a gantry of the computed tomography imaging system. In certain embodiments, the processing circuitry of the CT imaging system 10 is configured to generate a body contour of the subject based on the imaging data. In certain embodiments, the processing circuitry of the CT imaging system 10 is configured to automatically determine the reconstruction field of view based on the body contour.

In certain embodiments, the processing circuitry of the CT imaging system 10 is configured to automatically determine the reconstruction matrix parameters by automatically determine a matrix size based at least on the clinical task, the scanning parameters, and the reconstruction field of view. In certain embodiments, the processing circuitry of the CT imaging system 10 is configured to automatically determine the matrix size by utilizing a lookup table to determine the matrix size.

In certain embodiments, the processing circuitry of the CT imaging system 10 is configured to obtain additional selected parameters that influence the matrix size, wherein the matrix size is automatically determined based on the clinical task, the scanning parameters, the reconstruction field of view, and the additional selected parameters. In certain embodiments, the processing circuitry of the CT imaging system 10 is configured to automatically determine the matrix size by calculating the matrix size based on one or more of the scanning parameters and the additional selected parameters. In certain embodiments, the additional selected parameters are obtained via user input. In certain embodiments, the additional selected parameters are automatically determined, via the processor, based on the obtained clinical task. In certain embodiments, the additional selected parameters comprise reconstruction kernel, iterative reconstruction, and post processing filters.

In certain embodiments, the processing circuitry of the CT imaging system 10 is configured to automatically update a reconstruction prescription to include the reconstruction field of view and the matrix size. In certain embodiments, the processing circuitry of the CT imaging system 10 is configured to generate a reconstructed image utilizing the updated reconstruction prescription.

FIG. 5 is a flowchart of a method 60 for reconstructing CT imaging data. The method 60 may be performed by one or more components (e.g., processing circuitry) of the CT imaging system 10 in FIGS. 1-3. One or more steps of the method 60 may be performed simultaneously and/or in a different order than depicted in FIG. 5. One or more steps (and in some case all of the steps) of the method 60 may be performed automatically.

The method 60 includes obtaining/determining a clinical task for a scan of a subject (e.g., patient) with a CT imaging system (block 62). In certain embodiments, the clinical task is obtained (e.g., received) via user input. In certain embodiments, the clinical task is obtained (e.g., acquired) form a hospital information system or radiology information system. In certain embodiments, the clinical task is the purpose for the scan of the subject (e.g., detection of lesions, evaluation of vasculature, detection of bone fractures, etc.).

The method 60 also includes obtaining scanning parameters for the scan (block 64). Examples of scanning parameters include kVp, mA, rotation time, and helical pitch. The method 60 further includes automatically determining reconstruction matrix parameters for generating a reconstructed image from tomographic data obtained of the subject with the based at least on the clinical task and the scanning parameters (block 66). In certain embodiments, automatically determining the reconstruction matrix parameters includes automatically determining a reconstruction field of view (i.e., how much of a scan field of view is reconstructed into the image). In certain embodiments, the reconstruction field of view is determined utilizing a body contour of the subject determined by a 3D camera. In certain embodiments, the reconstruction field of view is determined utilizing a surface map of the subject derived from obtained LiDAR data. In certain embodiments, the reconstruction field of view is determined from an initial reconstructed image (of lower resolution or fidelity or image quality than the subsequent reconstruction image to be obtained) derived from initial tomographic data.

In certain embodiments, automatically determining the reconstruction matrix parameters includes automatically determining a matrix size based at least on the clinical task, the scanning parameters, and the reconstruction field of view. In certain embodiments, automatically determining the matrix size includes utilizing a lookup table to determine the matrix size. In certain embodiments, automatically determining the matrix size based on at least one or more of the scanning parameters.

The method 60 even further includes automatically updating a reconstruction prescription to include the reconstruction field of view and the matrix (block 68). The method 60 still further includes generating a reconstructed image utilizing the updated reconstruction prescription (block 70).

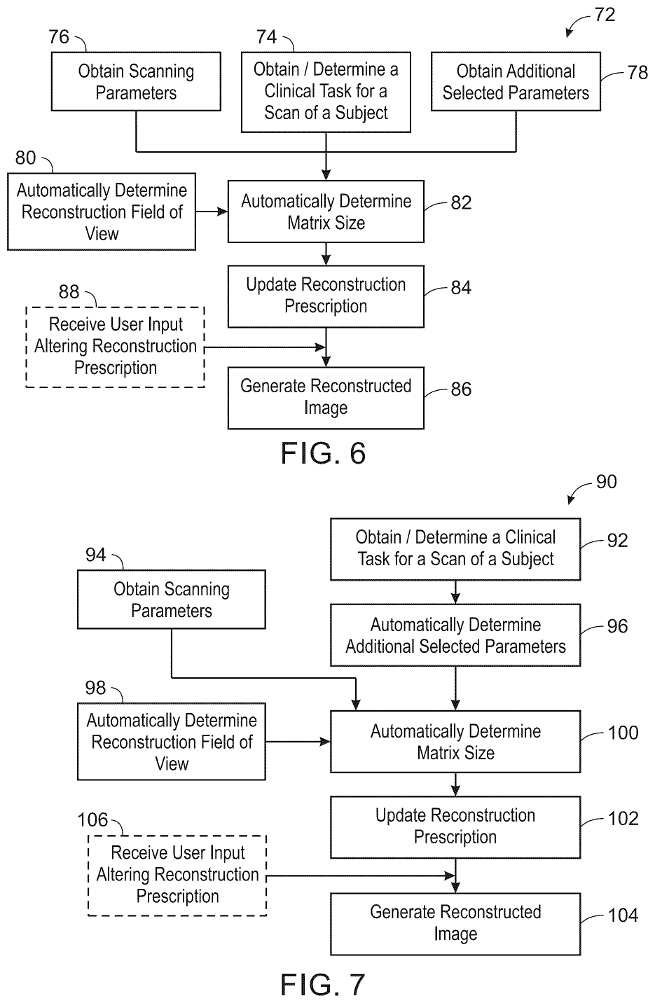

FIG. 6 is a flowchart of another method 72 for reconstructing CT imaging data. The method 72 may be performed by one or more components (e.g., processing circuitry) of the CT imaging system 10 in FIGS. 1-3. One or more steps of the method 72 may be performed simultaneously and/or in a different order than depicted in FIG. 6. One or more steps (and in some case all of the steps) of the method 72 may be performed automatically.

The method 72 includes obtaining/determining a clinical task for a scan of a subject (e.g., patient) with a CT imaging system (block 74). In certain embodiments, the clinical task is obtained (e.g., received) via user input. In certain embodiments, the clinical task is obtained (e.g., acquired) form a hospital information system or radiology information system. In certain embodiments, the clinical task is the purpose for the scan of the subject (e.g., detection of lesions, evaluation of vasculature, detection of bone fractures, etc.).

The method 72 also includes obtaining scanning parameters for the scan (block 76). Examples of scanning parameters include kVp, mA, rotation time, and helical pitch.

The method 72 further includes obtaining (e.g., receive) additional selected (e.g., user selected) parameters that influence the matrix size (block 78). Examples of additional selected parameters that influence matrix size include reconstruction kernel, iterative reconstruction, and post processing filters. In certain embodiments, the additional selected parameters are obtained via user input.

The method 72 even further includes automatically determining (e.g., selecting) a reconstruction field of view (block 80). In certain embodiments, the reconstruction field of view is determined utilizing a body contour of the subject determined by a 3D camera. In certain embodiments, the reconstruction field of view is determined utilizing a surface map of the subject derived from obtained LiDAR data. In certain embodiments, the reconstruction field of view is determined from an initial reconstructed image (of lower resolution or fidelity or image quality than the subsequent reconstruction image to be obtained) derived from initial tomographic data.

The method 72 still further includes automatically determining (e.g., selecting) a matrix size based at least on the clinical task, the scanning parameters, the reconstruction field of view, and the additional selected parameters (block 82). In certain embodiments, automatically determining the matrix size includes utilizing a lookup table to determine the matrix size. In certain embodiments, automatically determining the matrix size based on at least one or more of the scanning parameters and the additional selected parameters.

The method 72 even further includes automatically updating a reconstruction prescription to include the reconstruction field of view and the matrix (block 84). The method 72 still further includes generating a reconstructed image utilizing the updated reconstruction prescription (block 86). In certain embodiments, the method 72 includes receiving user input that alters the reconstruction prescription after it has been updated (block 88). This enables the user to manually change, if desired, the reconstructions matrix parameters (e.g., reconstruction field of view or matrix size) that were automatically determined.

FIG. 7 is a flowchart of a further method 90 for reconstructing CT imaging data. The method 72 may be performed by one or more components (e.g., processing circuitry) of the CT imaging system 10 in FIGS. 1-3. One or more steps of the method 90 may be performed simultaneously and/or in a different order than depicted in FIG. 7. One or more steps (and in some case all of the steps) of the method 90 may be performed automatically.

The method 90 includes obtaining/determining a clinical task for a scan of a subject (e.g., patient) with a CT imaging system (block 92). In certain embodiments, the clinical task is obtained (e.g., received) via user input. In certain embodiments, the clinical task is obtained (e.g., acquired) form a hospital information system or radiology information system. In certain embodiments, the clinical task is the purpose for the scan of the subject (e.g., detection of lesions, evaluation of vasculature, detection of bone fractures, etc.).

The method 90 also includes obtaining scanning parameters for the scan (block 94). Examples of scanning parameters include kVp, mA, rotation time, and helical pitch. The method 90 further includes automatically determining (e.g., selecting) additional selected parameters that influence the matrix size based on the obtained clinical task (block 96). Examples of additional selected parameters that influence matrix size include reconstruction kernel, iterative reconstruction, and post processing filters.

The method 90 even further includes automatically determining (e.g., selecting) a reconstruction field of view (block 98). In certain embodiments, the reconstruction field of view is determined utilizing a body contour of the subject determined by a 3D camera. In certain embodiments, the reconstruction field of view is determined utilizing a surface map of the subject derived from obtained LiDAR data. In certain embodiments, the reconstruction field of view is determined from an initial reconstructed image (of lower resolution or fidelity or image quality than the subsequent reconstruction image to be obtained) derived from initial tomographic data.

The method 90 still further includes automatically determining (e.g., selecting) a matrix size based at least on the clinical task, the scanning parameters, the reconstruction field of view, and the additional selected parameters (block 100). In certain embodiments, automatically determining the matrix size includes utilizing a lookup table to determine the matrix size. In certain embodiments, automatically determining the matrix size based on at least one or more of the scanning parameters and the additional selected parameters.

The method 90 even further includes automatically updating a reconstruction prescription to include the reconstruction field of view and the matrix (block 102). The method 90 still further includes generating a reconstructed image utilizing the updated reconstruction prescription (block 104). In certain embodiments, the method 90 includes receiving user input that alters the reconstruction prescription after it has been updated (block 106). This enables the user to manually change, if desired, the reconstructions matrix parameters (e.g., reconstruction field of view or matrix size) that were automatically determined.

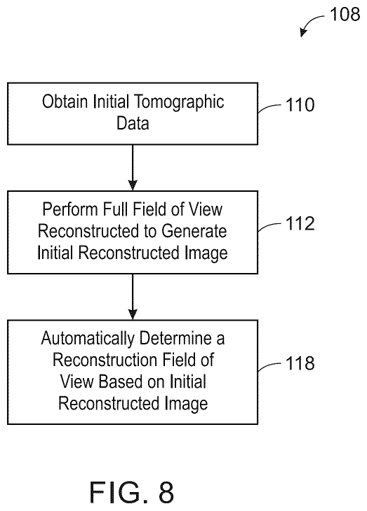

FIG. 8 is a flowchart of a further method 108 for determining a reconstruction field of view (e.g., utilizing an initial reconstructed image). The method 108 may be performed by one or more components (e.g., processing circuitry) of the CT imaging system 10 in FIGS. 1-3. One or more steps of the method 108 may be performed simultaneously and/or in a different order than depicted in FIG. 8. One or more steps (and in some case all of the steps) of the method 108 may be performed automatically.

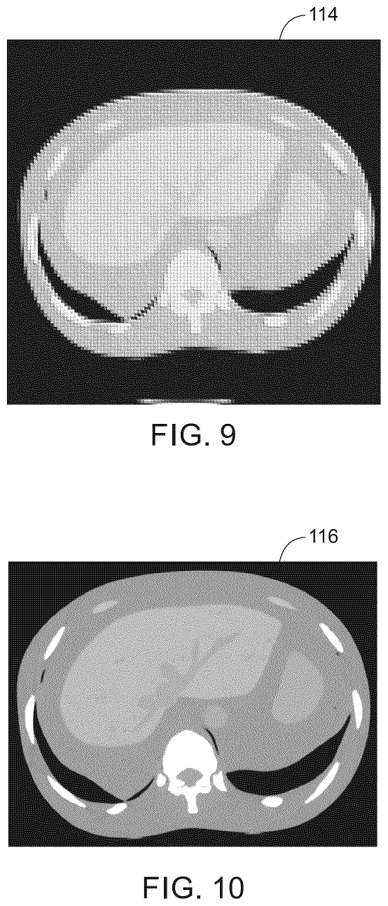

The method 108 includes obtaining (e.g., acquiring) initial tomographic data of the subject with the CT imaging system (block 110). The method 108 also includes performing full field of view reconstruction on the initial tomographic data to generate an initial reconstructed image (block 112). The initial reconstructed image has a lower resolution (e.g. lower fidelity or lower image quality) than a reconstructed image of the tomographic data generated during a subsequent scan utilizing the automatically determined matrix parameters as described in the method 60 in FIG. 5. The initial reconstructed image is not shown to the user. FIG. 9 depicts an example of an initial reconstructed image 114 (e.g., lower resolution reconstructed image) of a subject. FIG. 10 depicts an example of a reconstructed image 116 (e.g., higher resolution reconstructed image) of the subject that is based on tomographic data obtained during a subsequent scan utilizing the automatically determined matrix parameters. Returning to FIG. 8, the method 108 further includes automatically determining a reconstruction field of view based on the initial reconstructed image (block 118). For example, in certain embodiments, a computer algorithm may tailor the reconstruction field of view patient size based on the initial reconstructed image.

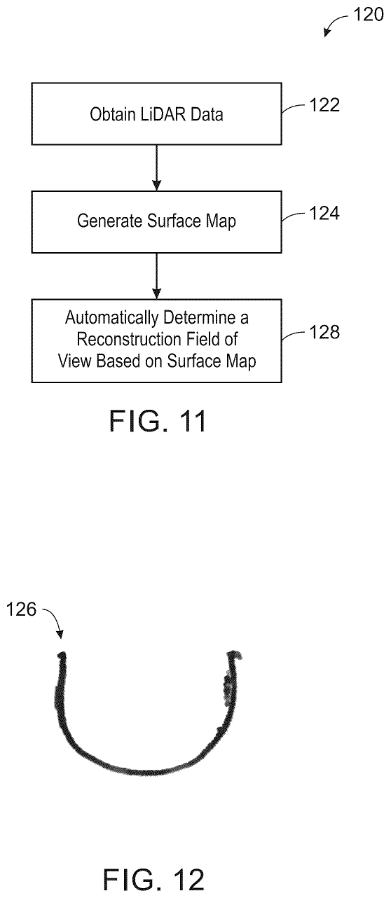

FIG. 11 is a flowchart of a further method 120 for determining a reconstruction field of view (e.g., utilizing surface map from LiDAR data). The method 120 may be performed by one or more components (e.g., processing circuitry) of the CT imaging system 10 in FIGS. 1-3. One or more steps of the method 120 may be performed simultaneously and/or in a different order than depicted in FIG. 11. One or more steps (and in some case all of the steps) of the method 120 may be performed automatically.

The method 120 includes obtaining LiDAR data of the subject acquired with a LiDAR scanning system coupled to a gantry of the CT imaging system (block 122). The method 120 also includes generating a surface map of the subject based on the LiDAR data (block 124). FIG. 12 depicts an example of a 2D contour representation 126 for a surface of the region of interest of a subject generated from LiDAR data. The method 120 further includes automatically determining a reconstruction field of view based on the surface map (block 128).

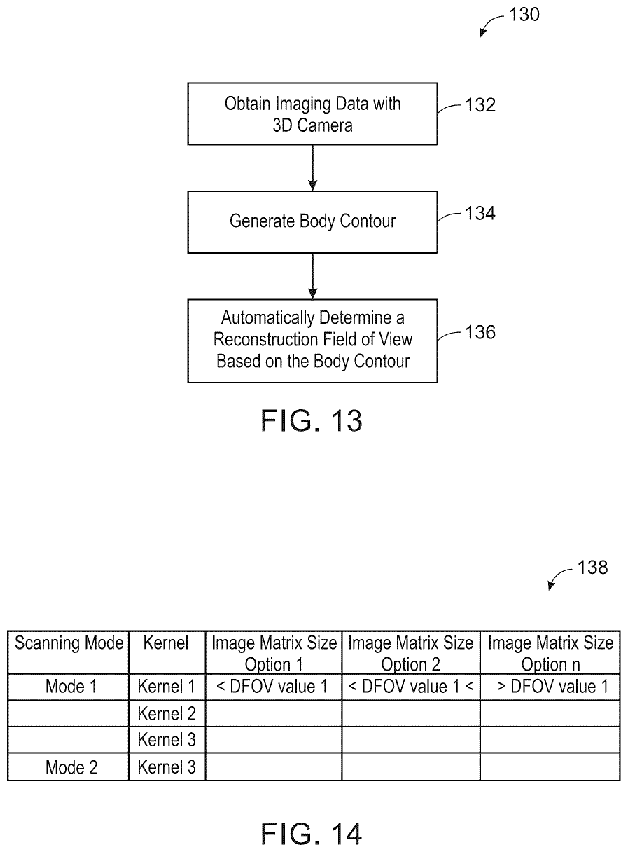

FIG. 13 is a flowchart of a further method 130 for determining a reconstruction field of view (e.g., utilizing a body contour). The method 130 may be performed by one or more components (e.g., processing circuitry) of the CT imaging system 10 in FIGS. 1-3. One or more steps of the method 130 may be performed simultaneously and/or in a different order than depicted in FIG. 13. One or more steps (and in some case all of the steps) of the method 130 may be performed automatically.

The method 130 includes obtaining imaging data of the subject acquired with a 3D camera coupled to a gantry of the CT imaging system (block 132). The method 130 also includes generating a body contour of the subject based on the imaging data (block 134). The method 130 further includes automatically determining a reconstruction field of view based on the body contour (block 136).

As mentioned above, in certain embodiments, automatically determining matrix size includes utilizing a lookup table to determine (e.g., select) the matrix size. FIG. 14 depicts an example of a lookup table 138 for determining matrix size. As depicted, the type of scanning mode, the kernel type, and reconstruction field of view are utilized on the lookup table 138 for determining the matrix size. In certain embodiments, other parameters may be utilized in conjunction with a lookup table to determine the matrix size. For example, other parameters may include focal spot and/or slice thickness. Alternatively, in certain embodiments, automatically determining the matrix size includes calculating, via the processor, the matrix size based on one or more of the scanning parameters and the additional selected parameters. For example, the configuration of the reconstruction kernel in conjunction with the determined reconstruction field of view to determine cutoff frequencies to determine (e.g., select) the matrix size.

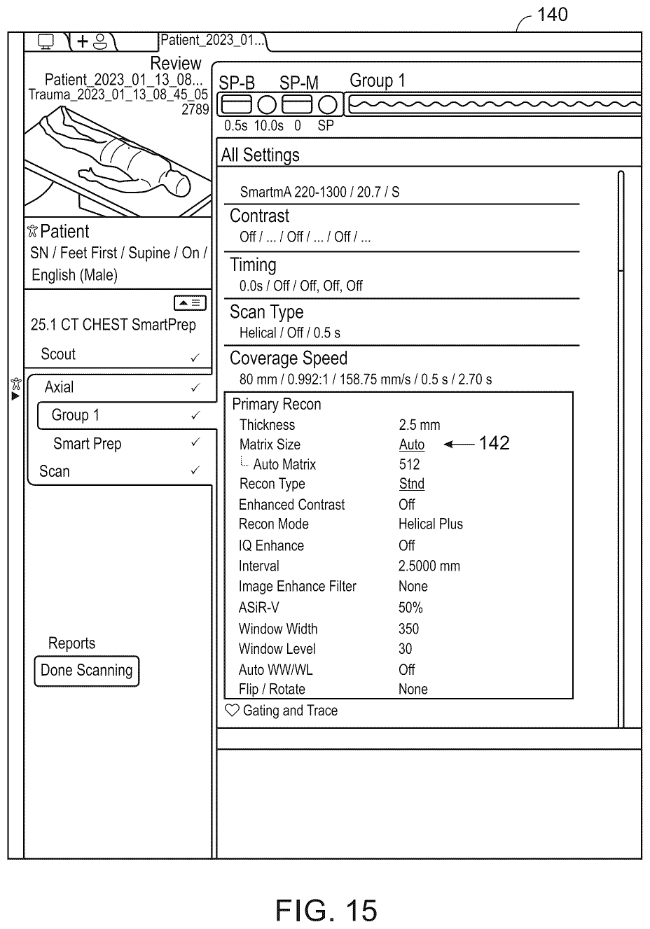

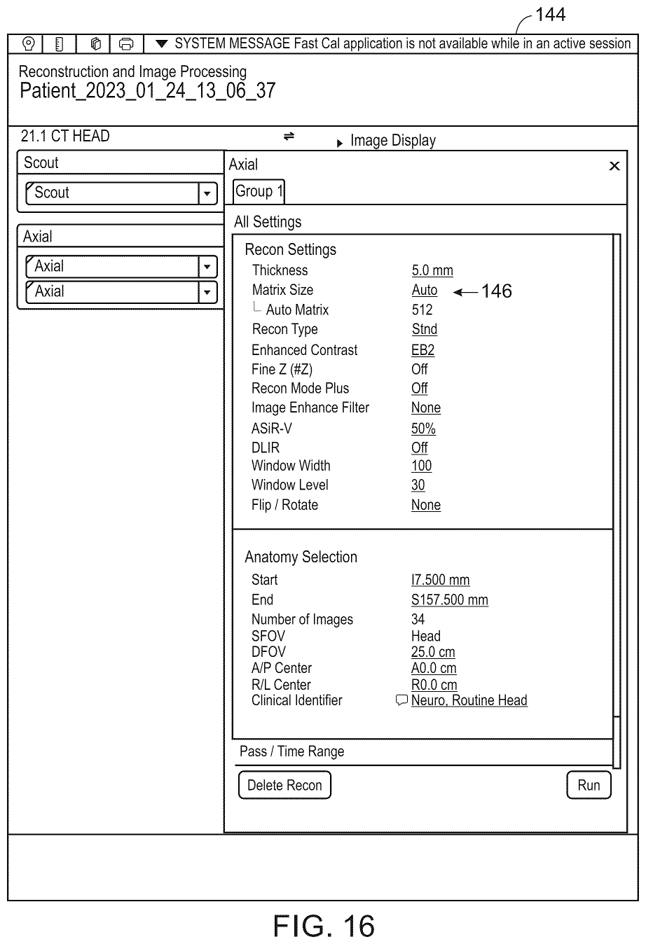

The automated image size feature may be utilized for both primary reconstruction (e.g., axial images from scanning) and secondary reconstruction (e.g., reconstructing orthogonal images from the primary reconstruction). FIG. 15 depicts a user interface 140 for conducting primary reconstruction utilizing the automated image size feature. As depicted, arrow 142 indicates the portion of the user interface indicating the enablement of the automatic image size feature and the automatically determined matrix size. FIG. 16 depicts a user interface 144 for conducting secondary reconstruction utilizing the automated image size feature. As depicted, arrow 146 indicates the portion of the user interface indicating the enablement of the automatic image size feature and the automatically determined matrix size. In certain embodiments, the user can override the automatic image size feature by manually selecting a matrix size.

Technical effects of the disclosed embodiments include providing for the automatic determination of reconstruction matrix parameters for generating a reconstructed image of optimal size. Technical effects of disclosed embodiments include enabling a user to benefit from using larger matrix sizes while minimizing their disk space utilization, thus, optimizing resource utilization. Technical effects of the disclosed embodiments further include enabling the utilization of a higher resolution CT scanner while not always generating large images. Technical effects of the disclosed embodiments further include enabling faster reconstruction. Technical effects of the disclosed embodiments even further include improving both the workflow and resource management without impacting image quality.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method, comprising:
obtaining, at a processor, a clinical task for a scan of a subject with a computed tomography imaging system;
obtaining, at the processor, scanning parameters for the scan; and
automatically determining, via the processor, reconstruction matrix parameters for generating a reconstructed image from tomographic data obtained of the subject with the scan based at least on the clinical task and the scanning parameters.

2. The computer-implemented method of claim 1, wherein automatically determining the reconstruction matrix parameters comprises automatically determining, via the processor, a reconstruction field of view.

3. The computer-implemented method of claim 2, further comprising:
obtaining, at the processor, initial tomographic data of the subject with the computed tomography imaging system;
performing, via the processor, full field of view reconstruction on the initial tomographic data to generate an initial reconstructed image, wherein the initial reconstructed image has a lower resolution than a reconstructed image of the tomographic data generated utilizing the reconstruction matrix parameters; and
automatically determining, via the processor, the reconstruction field of view based on the initial reconstructed image.

4. The computer-implemented method of claim 2, further comprising:
obtaining, at the processor, light detection and ranging (LiDAR) data of the subject acquired with a LiDAR scanning system coupled to a gantry of the computed tomography imaging system;
generating, via the processor, a surface map of the subject based on the LiDAR data; and
automatically determining, via the processor, the reconstruction field of view based on the surface map.

5. The computer-implemented method of claim 2, further comprising:
obtaining, at the processor, imaging data of the subject acquired with a three-dimensional camera coupled to a gantry of the computed tomography imaging system;
generating, via the processor, a body contour of the subject based on the imaging data; and
automatically determining, via the processor, the reconstruction field of view based on the body contour.

6. The computer-implemented method of claim 2, wherein automatically determining the reconstruction matrix parameters comprises automatically determining, via the processor, a matrix size based at least on the clinical task, the scanning parameters, and the reconstruction field of view.

7. The computer-implemented method of claim 6, wherein automatically determining the matrix size comprises utilizing, via the processor, a lookup table to determine the matrix size.

8. The computer-implemented method of claim 6, further comprising obtaining, at the processor, additional selected parameters that influence the matrix size, wherein the matrix size is automatically determined based on the clinical task, the scanning parameters, the reconstruction field of view, and the additional selected parameters.

9. The computer-implemented method of claim 8, wherein automatically determining the matrix size comprises calculating, via the processor, the matrix size based on one or more of the scanning parameters and the additional selected parameters.

10. The computer-implemented method of claim 8, wherein the additional selected parameters are obtained, at the processor, via user input.

11. The computer-implemented method of claim 8, wherein the additional selected parameters are automatically determined, via the processor, based on the obtained clinical task.

12. The computer-implemented method of claim 8, wherein the additional selected parameters comprise reconstruction kernel, iterative reconstruction, and post processing filters.

13. The computer-implemented method of claim 6, further comprising:
automatically updating, via the processor, a reconstruction prescription to include the reconstruction field of view and the matrix size; and
generating, via the processor, a reconstructed image utilizing the updated reconstruction prescription.

14. The computer-implemented method of claim 1, wherein the clinical task is obtained, at the processor, via user input.

15. The computer-implemented method of claim 1, wherein the clinical task is obtained, at the processor, from a hospital information system or radiology information system.

16. A system, comprising:
a memory encoding processor-executable routines; and
a processor configured to access the memory and to execute the processor-executable routines, wherein the processor-executable routines, when executed by the processor, cause the processor to:
obtain a clinical task for a scan of a subject with a computed tomography imaging system;
obtain scanning parameters for the scan; and
automatically determine reconstruction matrix parameters for generating a reconstructed image from tomographic data obtained of the subject with the scan based at least on the clinical task and the scanning parameters.

17. The system of claim 16, wherein automatically determining the reconstruction matrix parameters comprises automatically determining a reconstruction field of view.

18. The system of claim 17, wherein automatically determining the reconstruction matrix parameters comprises automatically determining a matrix size based at least on the clinical task, the scanning parameters, and the reconstruction field of view.

19. A non-transitory computer-readable medium, the non-transitory computer-readable medium comprising processor-executable code that when executed by a processor, causes the processor to:
obtain a clinical task for a scan of a subject with a computed tomography imaging system;
obtain scanning parameters for the scan; and
automatically determine reconstruction matrix parameters for generating a reconstructed image from tomographic data obtained of the subject with the scan based at least on the clinical task and the scanning parameters.

20. The non-transitory computer-readable medium of claim 19, wherein automatically determining the reconstruction matrix parameters comprises both automatically determining a reconstruction field of view and automatically determining a matrix size based at least on the clinical task, the scanning parameters, and the reconstruction field of view.
